(19) United States
(12) Patent Application Publication
(10) Pub. No.: US 2023/0332083 A1
(43) Pub. Date: Oct. 19, 2023

(54) FLUIDIZED CHAIN ELONGATION MEMBRANE BIOREACTOR FOR PRODUCTION AND RECOVERY OF CARBOXYLATES FROM ORGANIC BIOMASS

(71) Applicant: King Abdullah University of Science and Technology, Thuwal (SA)

(72) Inventors: Pascal Saikaly, Thuwal (SA); Jiajie Xu, Thuwal (SA); Bin Bian, Thuwal (SA); Muhammad Ali, Thuwal (SA)

(21) Appl. No.: 18/044,522

(22) PCT Filed: Sep. 9, 2021
(86) PCT No.: PCT/IB2021/058221

§ 371 Date: Mar. 8, 2023

Related U.S. Application Data
(60) Provisional application No. 63/076,266, filed on Sep. 9, 2020.

(51) Int. Cl. C12M 1/12; C12M 1/00; C12P 7/52; C12P 7/6409

(52) U.S. Cl. CPC C12M 25/12; C12M 47/10; C12P 7/52; C12P 7/6409

(57) ABSTRACT

Bioreactors for production and recovery of medium chain carboxylates from organic biomass are disclosed. Methods for improved production and recovery of medium chain carboxylates from organic biomass are also disclosed. The bioreactors can be used as a chain-elongation bioreactor, and a method of use thereof results in improved production and recovery of medium chain carboxylates from organic biomass. The bioreactor includes a shell defined by one or more walls and a length, and a plurality of porous hollow fiber membranes placed inside the reactor for continuous liquid-liquid extraction, as well as granular activated carbon (GAC) as biocarriers. The plurality of hollow fiber membranes is mounted such that a percentage of the length of the shell remains unoccupied by the plurality of porous hollow fiber membranes.

### CROSS REFERENCE TO RELATED APPLICATION

This application claims the benefit of and priority to U.S. Provisional Application No. 63/076,266, filed Sep. 9, 2020, which is hereby incorporated herein by reference in its entirety.

### FIELD OF THE INVENTION

This invention relates to an improved process for production and recovery of medium chain carboxylates from organic biomass.

### BACKGROUND OF THE INVENTION

Carbon recovery from organic waste or wastewater reduces the cost of waste treatment and also increases recoverable chemical energy (Hao, et al., Water Res. 2019, 161, 74-77; Lu, et al., Nat. Sustain 2018, 1, 750-758). One of the biotechnologies that is of interest for renewable chemical production is the carbon chain elongation platform. Carbon chain elongation platform harnesses the potential of certain microbes in anaerobic fermentation biotechnology to generate medium-chain carboxylic acids (MCCAs, C6-C12) from short-chain carboxylic acids (SCCAs, C2-C5) and an electron donor (e.g., ethanol), which can be obtained through the hydrolysis of organic biomass (Angenent, et al., Environ. Sci. Technology 2016, 50, 2796-2810; Daly, et al., ACS Sustain Chem. Eng. 2020, 8, 13934-13944; Xu, et al., Joule 2018, 2, 280-295). The pathway of reverse β-oxidation is considered a thermodynamically favorable microbial synthesis pathway to produce MCCAs (Dellomonaco, et al., Nature 2011, 476, 355-359; González-Cabaleiro, et al., Energy Environ. Sci. 2013, 6, 378003789). MCCAs are valuable molecules and could be utilized for various industrial and agricultural applications, such as sustainable antimicrobials (Kim and 30 Rhee, Appl. Environ. Microbiol. 2013, 79, 6552-6560), precursors for liquid biofuel production (Urban, et al., Energy. Environ. Sci. 2017, 10, 2231-2244), oleochemical production (Zhu, et al., Nat. Catal. 2020, 3, 64-74), and livestock feed additives for growth (Mills, et al., J. Dairy Sci. 2010, 93, 4262-4273). However, it is challenging to reach a high concentration of MCCAs in the microbial synthesis system due to the cellular toxicity of MCCAs (Zhu, et al., Nat. Catal. 2020, 3, 64-74). The uncharged carboxylic acids disrupt cell membrane. Further, these acids with longer carbon chains are more toxic due to the increased hydrophobicity of the carbon chain (Butkus, et al., Appl. Environ. Microbiol. 2011, 77, 363-366; Harroff, et al., Environ. Sci. Technol. 2017, 51, 9729-9738). Currently, in-line extraction system for MCCAs is considered one of the best options for reducing cell membrane toxicity and end-product feedback inhibition, thus enabling high

MCCAs production rates (Lambrecht, et al., Microb. Cell Fact. 2019, 18, 1-17; Michel-Savin, et al., Appl. Microbiol. Biotechnol. 1990, 33, 127-131; Roe, et al., Microbiology 2002, 148, 2215-2222).

Several technologies have been applied for MCCA in-line extraction directly from a fermentation broth, including electrodialysis cell (López-Garzón and Straathof, Biotechnol. Adv. 2014, 32, 873-904; Wang, et al., Bioresour. Technol. 2013, 147, 442-448), permeate membrane (Zhu, et al., ACS EST Eng. 2021, 1, 141-153), electrolysis unit (Carvajal-Arroyo, et al., Chem. Eng. J. 2020, 416, 127886), electrodialysis/phase separation cell (Xu, et al., Environ. Sci. Technol. 2021, 55, 634-644), and membrane-based liquid-liquid extraction (i.e., pertraction) (Gehring, et al., J. Chem. Technol. Biotechnol. 2020, 95, 3105-3116; Ge, et al., Environ. Sci. Technol. 2015, 49, 8012-8021; Saboe, et al., Green. Chem. 2018, 20, 179101804; Xu, et al., Chem. Commun. 2015, 51, 6847-6850). Pertraction for in-line extraction of MCCAs has been well studied and has already been applied in a pilot-scale system (CaproX) due to its low energy cost (mainly requiring electric power to pump the fermentation broth, hydrophobic solvent and pertraction solution) and selective extraction of the longest possible carbon chain of carboxylate (Angenent, et al., Bioresour. Technol. 2018, 247, 1085-1094). The driving force for MCCA pertraction is a pH gradient (˜5.0 to ˜9.0) to specifically extract undissociated carboxylic acids by diffusion through a forward and a backward membrane (Angenent, et al., Bioresour. Technol. 2018, 1085-1094). In accordance with previous pertraction mass transfer model study (Kucek, et al., Energy Environ. Sci. 2016b, 9, 3482-3494), an increase in the recycle flow rates of fermentation broth (0-225 m ^{d-1}) led to an increase in the MCCA mass transfer rate. However, increasing the recycle low rates of the extractant (e.g. hydrophobic solvent) or the pertraction solution (e.g. alkaline extraction solution) did not affect the overall mass transfer rates, indicating that mass transfer limitations were at the interface of the fermentation broth and the hydrophobic membrane contactor.

There is still a need for improved methods for production and recovery of medium chain carboxylates from organic biomass.

Accordingly, it is an object of the present invention to provide a system and method for organic biomass conversion into MCCAs system that permit more effective biological conversion of food waste as well as wastewater, and that reduces associated energy use and maintenance requirements.

### SUMMARY OF THE INVENTION

Bioreactors for production and recovery of medium chain carboxylates from organic biomass are disclosed. Methods for improved production and recovery of medium chain carboxylates from organic biomass are also disclosed. The bioreactors can be used as a chain-elongation bioreactor, and a method of use thereof results in improved production and recovery of medium chain carboxylates from organic biomass. The bioreactor includes a shell defined by one or more walls and a length, and a submerged membrane, preferably a plurality of porous hollow fiber (HF) membranes placed inside the shell for continuous liquid-liquid extraction, as well as granular activated carbon (GAC) as biocarriers. The plurality of hollow fiber membranes is mounted such that a percentage of the length of the shell (e.g., between about 10% and about 70%) remains unoccupied by the plurality of porous hollow fiber membranes. In some preferred embodiments, the size of GAC is from about 0.5 to about 1.5 mm.

The GAC particles play a bi-functional role: 1) increase biomass concentration in the reactor; and 2) reduce membrane fouling; thus enhancing MCCA yield and lowering operational cost. The disclosed method, by addressing these two bottlenecks, result in improved product generation rates and yields.

The disclosed bioreactor and methods can be used in food waste treatment, high chemical oxygen demand (COD) wastewater treatment and bioenergy conversion. The disclosed bioreactor is used to improve methods for producing and sequestering carboxylates (e.g., C3 to C8 carboxylates or C6 to C12) from biomass using microorganisms.

### BRIEF DESCRIPTION OF THE DRAWINGS

FIGS. 1A and 1B show schematics of two pertraction strategies during Periods I to IX (Table 1): FIG. 1A shows a pertraction system using only internal hollow fiber membrane to extract MCCAs during Periods I to VI. Biogas recirculation was applied during Periods II to VI. FIG. 1B shows a pertraction system using internal and external hollow fiber membrane simultaneously to extract MCCAs during Periods VII to IX. Biogas recirculation was applied during Period VII. Broth recirculation was applied during Periods VIII to IX. HF: Hollow Fiber. Dash line represents the gas flow and solid line represents the liquid flow. FIG. 1C. shows hydraulic Retention Time (HRT) and loading rate during Periods I to IX. The blue line represents the HRT and the orange line represents the loading rate. FIG. 1D is a graph showing Carboxylate mass transfer coefficient with abiotic synthetic broth during Stage A and B. C2: acetic acid; C4: n-butyric acid; C6: n-caproic acid; C8: n-caprylic acid. FIG. 1E is a graph showing solid concentrations during Periods I to IX. The blue line represents the total solid concentration in the effluent. The orange line represents the volatile solid concentration in the effluent.

FIGS. 2A-2C show carboxylic acids concentration in the bioreactor broth and biogas production during Periods I to IX. FIG. 2A is a stacked area chart for broth concentration of carboxylic acids (cumulative). FIG. 2B is a stacked area chart for a production rate of carboxylic acids including effluent, internal extraction and external extraction (cumulative). FIG. 2C is a line chart for biogas production rate (non-cumulative). FIG. 2D. shows ethanol concentration in the effluent during Periods I to IX.

FIG. 3 is a heatmap of relative OTU abundances of the nine microbiome samples collected during Periods Ito IX. The top 20 OTUs with relative abundance ≥1% for one or more of the microbiome samples are listed. The OTUs are classified down to the lowest taxonomic level (o: order, f: family, g: genus) possible.

### DETAILED DESCRIPTION OF THE INVENTION

### I. Definitions

The term “anaerobic ferminatation” is used herein to mean a fermentation carried out under anaerobic conditions by eukaryolic or prokaryotic microorganisms, such as bacteria, fungi, algae or yeasts.

“Broth,” refers to the stream or media in a bioreactor containing a compound to be extracted. The compound can be a medium chain fatty acid (MCCA).

“Shell volume” refers to the volume of space enclosed by the shell of the bioreactor described herein.

### II. Bioreactors

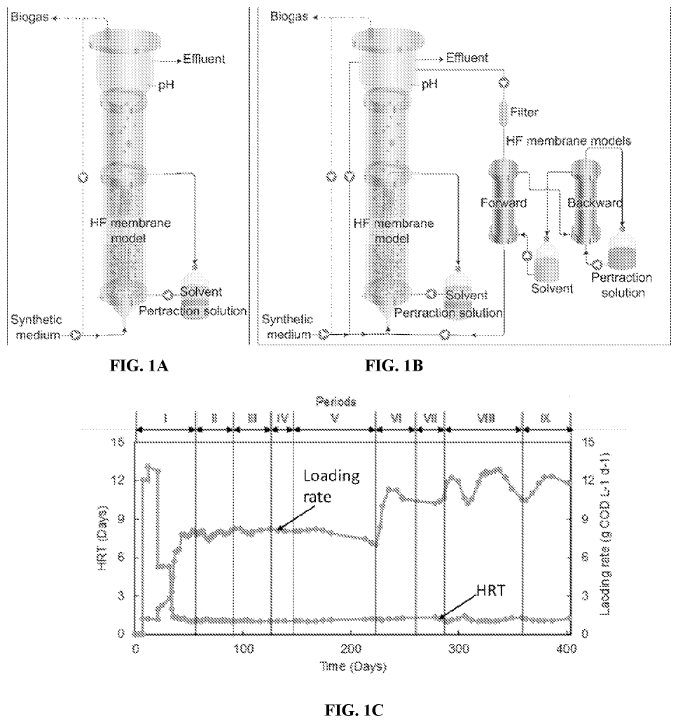

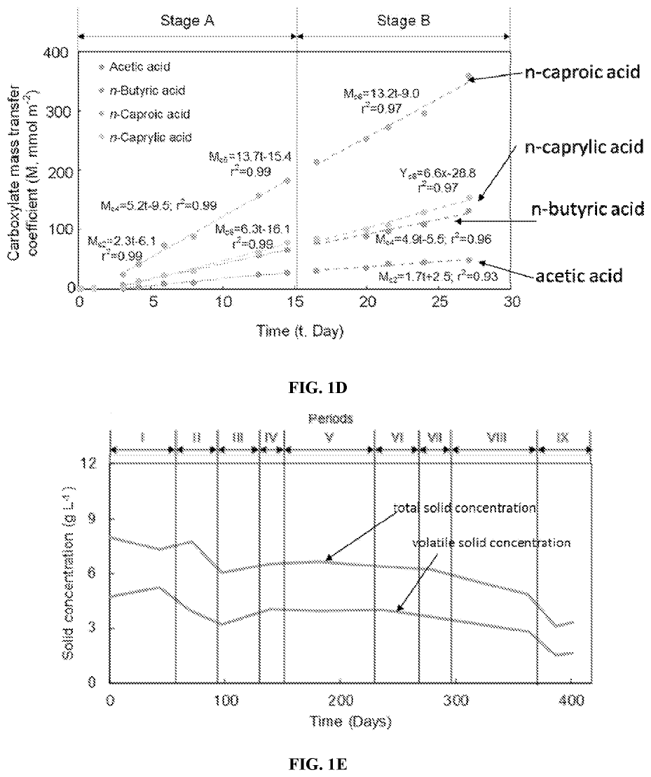

The present invention provides a bioreactor (FIG. 1A) containing a shell and a submerged membrane module that is placed inside the shell for continuous liquid-liquid extraction. The shell is defined by one or more walls. Preferably, the submerged membrane module contains a plurality of hollow fiber membranes. Preferably, hollow fibers in the plurality of hollow fiber membranes are porous. The plurality of hollow fiber membranes does not span the entire length of the shell, such that a length of between about 20% and about 50% of the length of the shell, remains unoccupied by the plurality of hollow fiber membranes. The inside of the shell can also contain granular activated carbon (GAC) as bio-carriers. GAC is used to control membrane fouling. GAC also possesses high surface area for colonization by microbes in the bioreactor. Thus, the GAC particles are expected to serve as bio-carriers for enhancing the colonization of chain-elongating thermophilic microbes in AnFMBR. The bioreactor preferably does not include a forward or backward HF membrane module (FIG. 1B).

The present invention has three advantages: 1) reduce footprint of the extraction system and 2) increase biomass concentration in the reactor and 3) reduce membrane fouling.

The chain-elongation carboxylates system disclosed herein is characterized in that it combines a fluidized bed bioreactor with membrane-based liquid-liquid extraction. It includes a bioreactor including active chain-elongation organisms; fluidized particles which is support media to be attached by the chain-elongation organisms; and membranes including a submerged membrane module and back extraction membrane module. The fluidized particles come into direct contact with the submerged membrane.

### A. Shell

i. Materials

The shell of the bioreactors disclosed herein can be made from any material that provides sufficient strength and dimensional stability for carrying out the desired mass transfer operations. Examples of suitable materials include polypropylene, polyvinylidene fluoride, polyvinyl chloride, metals (such as silver, zinc, copper, aluminum, nickel, iron, titanium, and chromium), metal alloys of any of the preceding metals, ceramics, glass, borosilicate-tempered glass, steel (e.g., stainless steel, carbon steel, etc), plastics (e.g., epoxy resins, UV cured resins, thermosetting resins, etc), ceramics, composites, quartz, silicon, and combinations thereof.

ii. Shape The shell can have a variety of different shapes, such as a cylinder, rectangle, square, pentagon, hexagon, octagon, etc. In some preferred forms, the shell has a cylindrical shape.
iii. Size

The design of the bioreactor is not limited by volumetric size, i.e., as determined by the dimensions of shell. For instance, the bioreactor can be an industrial scale reactor or a laboratory scale reactor. Laboratory scale reactors typically have shell volumes in the range of a few millimeters (e.g. 2 mL) to a few liters (e.g. 1 L, 2 L, 2.25 L, 3 L, or 5 L). In some forms, bioreactor volume is between 1 L and 5 L, such as 2.25 mL. In some forms, the bioreactor volume is between 0.1 m³ and 300 m³, such as 0.5 m³, from 0.45 m³ to 0.60 m³, 0.50 m³ to 0.60 m³, 1.0 m³, 2.0 m³, 3.0 m³, 4.0 m³, 5.0 m³, 10.0 m³, 20.0 m³, 25.0 m³, 50.0 m³, 75.0 m³, 100.0 m^{3,} etc.

Where the shell is a cylinder, the cylinder can have an internal diameter between 3 cm and 10 cm, such as 5.5 cm. The cylinder can have height between 50 cm and 150 cm, such as 95 cm. In some forms, shell is a cylinder with a diameter of about 5.5 cm and a height of about 95 cm.

B. Hollow Fiber Membrane The hollow fiber membranes for use in the disclosed bioreactors can be hydrophobic, hydrophilic, or a composite of both. Preferably, the hollow fibers are porous. In liquid/liquid extraction systems, low membrane mass transfer resistance can be obtained if the pores of the hollow fiber membranes contain a fluid in which the compound to be extracted is very soluble. Thus, a hydrophilic membrane or hydrophobic membrane can be used when the compound to be extracted is hydrophilic or hydrophobic, respectively.
i. Materials

The hollow fiber membranes disclosed herein, can be made from polymeric materials, non-polymeric materials, or a combination thereof. Materials for the hollow fiber membranes include, but are not limited, cellulose (e.g., regenerated cellulose), cellulose acetate, polysulfone, polyacrylonitrile, inorganic carbon, alumina, polypropylene, polyethylene, polyvinylidene fluoride, polytetrafluoroethylene, polyether sulfone, sulfonated polyether sulfone, and a combination thereof

ii. Size

The hollow fiber membranes can have lengths that are suitable for a given mass transfer process. However, the lengths can be limited by the dimensions of the shell, the pumping costs that could be incurred by increasing the lengths of the hollow fiber membranes, or a combination thereof. Suitable lengths are between 5 cm and 50 cm, such as 44 cm; between 18 cm and 120 cm, between 18 cm and 185 cm, between 25 cm and 310 cm, between 60 and 110 cm, or a combination thereof The lengths of the hollow fiber membranes can be, independent of the lengths of other hollow fiber membranes in the bioreactor, the same or different. In some forms, all the hollow fiber membranes have the same length. In some forms, the lengths of the hollow fiber membranes have a Gaussian distribution.

The hollow fiber membranes can have internal diameters that are suitable for a given mass transfer process. Suitable internal diameters can be between 0.1 mm and 10 mm, such as between 0.20 mm and 3 mm, between 0.5 mm and 3.5 mm, between 0.1 mm and 6 mm, between 0.5 mm and 1.5 mm. The internal diameters of the hollow fiber membranes can be, independent of the internal diameters of other hollow fiber membranes in the bioreactor, the same or different. In some forms, all the hollow fiber membranes have the same internal diameter. In some forms, the internal diameters of the hollow fiber membranes have a Gaussian distribution.

The hollow fiber membranes can have wall thicknesses that are suitable for a given mass transfer process. Suitable wall thickness can be between 10 μm and 1 mm, such as between 30 μm and 0.5 mm. In some forms, the wall thickness is uniform over the length of the hollow fiber membranes. The wall thicknesses of the hollow fiber membranes can be, independent of the wall thicknesses of other hollow fiber membranes in the bioreactor, the same or different. In some forms, all the hollow fiber membranes have the same wall thickness. In some forms, the wall thicknesses of the hollow fiber membranes have a Gaussian distribution.

iii. Spacing/Density

Preferably, a plurality of hollow fiber membranes is assembled, i.e., potted, and mounted into the bioreactor's shell. Suitable materials for potting the hollow fiber membranes include polyepoxides (such as solvent-resistant polyepoxides), polyurethane, polypropylene, or a combination thereof. The hollow fiber membranes can be uniformly or non-uniformly distributed inside the shell. For instance, to obtain uniform spacing, the hollow fibers membranes can be woven into a fabric, potted, and mounted into the shell. In some forms, hollow fiber membranes are arranged in configurations such as cylindrical tube bundles, helically wound bundles, rectangular bed of fibers, or a combination thereof.

When a plurality of hollow fiber membranes is mounted into the shell, the packing density of the hollow fiber membranes preferably provides efficient fluidization of the hollow fiber membranes, which can give rise to high mass transfer rates. The packing density is the ratio of volume occupied by the hollow fiber membranes to the internal volume of the shell. In some forms, the packing density is at least 10% and less than 80%, such as 10%, 15%, 20%, 25%, 30%, 35%, 40%, 45%, 50%, 55%, 60%, 65%, and 70%.

The number of hollow fiber membranes can be selected, such that the fibers have a suitable interfacial area with the broth containing the compound to be extracted, a suitable volume utilization, or a combination thereof.

The plurality of hollow fiber membranes does not span the entire length of the shell, such that a percentage of the length of the shell remains unoccupied by the plurality of hollow fiber membranes. For example, one end of the plurality of hollow fiber membranes is mounted at a first end of the shell, while the other end of the plurality of hollow fiber membranes is mounted towards a second end of the shell, such that a length between about 10% and about 70%, between about 10% and about 60%, between about 10% and about 50%, between about 20% and about 50%, or between about 20% and about 30%, of the length of the shell, as measured from the second end, is left unoccupied by the plurality of hollow fiber membrane. For example, in some forms, one end of the plurality of hollow fiber membranes is mounted at a first end of the shell, while the other end of the plurality of hollow fiber membranes is mounted at the middle of the shell, i.e., about 50% the length of the shell, as measured from the second end, is left unoccupied by the plurality of hollow fiber membrane.

iv. Pore Sizes

The hollow fiber membranes can have pore sizes that are suitable for a given mass transfer process. Suitable pore sizes can be between 0.1 μm and 5 μm, such as between 0.1 μm and 0.2 μm, between 0.1 μtm and 0.4 μm, between 0.1 μm and 0.65 μm, between 0.1 μm and 1 μm, between 0.2 μm and 0.4 μm, between 0.2 μm and 0.65 μm, between 0.4 μm and 0.65 μm, or a combination thereof In some forms, the pore sizes uniform over the length of the hollow fiber membranes. The pore sizes of the hollow fiber membranes can be, independent of the pore sizes of other hollow fiber membranes in the bioreactor, the same or different. In some forms, all the hollow fiber membranes have the same porosity.

C. Solvent through Hollow Channel of Hollow Fiber When the bioreactor is being used, one or more solvents flow through the hollow channel of a plurality of the hollow fiber membranes. Preferably, the hollow channel extends axially, i.e., along the length of the hollow fiber membrane, from one end to another end. In some forms, the one or more solvents are organic solvents. In some forms, the organic solvents are hydrophobic solvents. Suitable solvents include, but are not limited to, mineral oil solvent with tri-n-octylphosphine oxide (e.g., mineral oil solvent 15 with 3% tri-n-octylphosphine oxide), N-methylpyrrolidone, methyl isobutyl ketone, xylene, n-butanol, 1,2-butanediol, and a combination thereof.

### D. Pertraction Solution

In liquid/liquid extraction systems, extraction of the compound that was extracted into the solvent flowing axially through the hollow fiber membranes often requires a second separation step. The second separation step can involve a solution, referred to herein as a pertraction solution. The pertraction solution contacts the solvent from the hollow fiber membranes and preferably remains phase-separated from the solvent. Preferably, the pertraction solution and solvent from the hollow fiber membranes are in direct contact, i.e., not separated by a membrane. During this phase-separated contact, the extracted compound(s) are stripped from the solvent from the hollow fiber membranes into the pertraction solution. In a scenario where the compound(s) extracted were carboxylic acids, the pertraction solution is an aqueous phase. Preferably, the pertraction solution is maintained at an alkaline pH, such as between 8 and 14, between 9 and 13, or between 9 and 11. Accordingly, the pertraction solution can contain a base (e.g., an inorganic base) such as sodium hydroxide or hydrogen carbonates, such as sodium hydrogen carbonate. In some forms, the pertraction solution can also contain small amounts of an acid (e.g. 0.2 M boric acid), but the overall pH is alkaline. For example, the pertraction solution can contain 0.2 M boric acid and 2 M sodium hydroxide solution. Preferably, the chemical components (e.g., bases) in the pertraction solution do not diffuse into the solvent from the hollow fibers, such that the extraction capability of the solvent remains stable.

### E. Materials to Sequester Microorganisms

The inside of the shell can also contain materials to sequester microorganisms. These materials are known as biocarriers. Biocarriers are generally inert, porous, and can sequester, retain, and enhance the number of microorganisms within their structure.

The biocarriers may be sand, granular activated carbon (GAC), glass, polystyrene beads, plastic materials of polypropylene, polyethylene, polyvinyl dichloride, polytetrafluoroethylene, latex, rubber, agarose, or other materials as commonly used in traditional fluidize bed reactors. The size of GAC can be between about 0.5 mm and about 1.5 mm. The GAC of this size is effective in both colonizing organism and holding particulate matter, and prevent membrane clogging of MCCAs passing into organic solvent. Both the of submerged and back extraction membrane module are preferably hollow fiber (HF) membranes. The submerged HF membranes permit MCCAs in broth to go through membrane pore into organic solvent, but not the organisms, and broth, and prevents the organic solvent from flowing out through the pores of the HF membranes.

### III. Methods of Use

The disclosed bioreactor and methods can be used in food waste treatment, high COD wastewater treatment and bioenergy converting. Various sources of carbohydrate containing biomass can be used. For example, carbohydrate containing biomass can be municipal waste (food, yard, paper, organic fraction of source-sorted garbage, wood or biomass-based building materials, compost feedstocks), animal waste, agricultural residues (e.g., corn stover, corn fiber, wheat, barley, or rye straw, hay, silage, fruit or vegetable processing wastes), by-products of alternative energy processes (corn beer, sugar cane bagasse, butanol beer), wood wastes (e.g., saw mill, paper wastes, wooden pallets, building materials), biosolids wastes (waste activated sludge), animal hydrolysates (dead animals made soluble), aste from food production, such as cheese whey, yogurt production waste, beer production waste (including spent grain), or animal rendering waste. Waste carbon, including organic waste, wastewater, CO₂ and syngas converts into short carboxylates. Those short carboxylates can convert into medium chain carboxylates using the disclosed bioreactor and methods which use a microbial mixture packaged in fluidized particles.

The disclosed bioreactor is used to improve methods for producing and sequestering carboxylates (e.g., C3 to C8 carboxylates or C6 to C12 carboxylates) from biomass using microorganisms., via chain elongation.

Chain elongation is an open-culture biotechnological process which converts short chain fatty acids and an electron donor to medium chain fatty acids (MCFAs). Carbon chain elongation platform harnesses the potential of certain microbes in anaerobic fermentation biotechnology to generate medium-chain carboxylic acids (MCCAs, C6-C12) from short-chain carboxylic acids (SCCAs, C2-C5) and an electron donor (e.g., ethanol), which can be obtained through the hydrolysis of organic biomass. MCCAs are produced by certain bacteria in a strongly reduced anaerobic environment, via a metabolic pathway that has been recently reviewed by Spirito et al. [29]. The bacteria gain energy by combining the oxidation of an electron donor, i.e., lactic acid or ethanol, to acetyl-CoA with the reductive elongation of acetyl-CoA with acetic acid (C2), propionic acid (C3), butyric acid (C4), pentanoic acid (C5), or caproic acid (C6) generating a carboxylic acid with 2 additional carbons at each step.

Useful microorganisms include, but are not limited to those that effect chain elongation. Examples include Clostridium strains producing butyric acid and Megasphaera hexarioica producing caproic acid from the butyric acid. Clostridium kluyveri was the first isolated bacterium capable of producing caproic acid. Acidic pH are not favourable for growth of known ethanol chain elongators: the type-strain of C. kluyveri, strain DSM555, has an optimum pH of 6.4, and grows in a pH range between 6 and 7.5. Another, more recent, isolate obtained from bovine rumen—strain 3231B—has been demonstrated to grow at pH as low as 4.88, although the optimal pH for growth of this strain also lies between pH 6.4 and 7.6. Biological production of hexanoic acid has been reported for a few strict anaerobic bacteria. Clostridium kluyveri produced hexanoic acid from ethanol, a mixture of cellulose and ethanol [5] and from ethanol and acetate. Strain BS-1, classified as a Clostridium cluster IV, produced hexanoic acid when cultured on galactitol. Megasphaera elsdenii produced a diverse mixture of carboxylic acids such as formic acid, acetic acid, propionic acid, butyric acid, pentanoic acid, and hexanoic acid from glucose and lactate and sucrose and butyrate. It is postulated that hexanoic acid is produced by two consecutive condensation reactions: the first is the formation of butyric acid from two acetyl-CoAs, and the second is the formation of hexanoic from one butyryl-CoA and one acetyl-CoA. The condensation reaction of two acetyl-CoAs to butyric acid has been well reported in Clostridium spp. such as Clostridium pasteurianum, C. acetobutylilcyn, and C. kluyveri. (reviewed in Jeon, et al., Biotechnology for Biofuels volume 9, 129 (2016) htips://doi.org/10.1186/s:13068-0164)549-3.

In some embodiments, the disclosed methods use mixed microbial culture (MCC). MMC use the synergy of bio-catalytic activities from different microorganisms to transform complex organic feedstock, such as by-products from food production and food waste. In the absence of oxygen, the feedstock can be converted into biogas through the established anaerobic digestion (AD) approach. (reviewed in Groof, et al., Molecules 2019, 24, 398; doi:10.3390/molecules24030398).

### A. Types of Flow, Flow Rates, pH, and Temperature

In use, a broth flows on the shell side of the bioreactor, and is in contact with the external surfaces of the hollow fiber membranes. Further, as solvent flows axially through the hollow channel of a plurality of the hollow fiber membranes. The solvent and the broth are separated by an interface formed by the walls of the hollow fiber membranes. As the broth flows over the hollow fiber membranes, a compound to be extracted from the broth diffuses across the membrane into the solvent.

The broth can be produced when an inlet stream flows into the bioreactor, and microorganisms metabolize one or more components in the inlet stream to produce a compound to be extracted. The inlet stream can also be the broth that already contains the compound to be extracted. As the inlet stream flows through the bioreactor and contacts the plurality of hollow fibers, (i) microorganisms (when present) in the bioreactor convert a component of the inlet stream into a product and/or a chemical compound to be extracted, and/or (ii) a compound is extracted from the inlet stream across the plurality of hollow fiber membranes. Thus, within the bioreactor, the inlet stream is generally a combination of some or all of its initial components and/or products. However, for simplicity, the inlet stream modified within the bioreactor, as described herein, is referred to as the shell side stream. Preferably, (i) the inlet fluid flows continuously into the bioreactor; (ii) the compound is extracted continuously; (iii) the solvent flows continuously through the hollow channels of the hollow fiber membranes; (iv) an outlet stream (for example an effluent) continuously exits the bioreactor; or a combination of (i), (ii), (iii), and (iv), such as (i)-(iv).

In some forms, the broth and the solvent flowing axially in one or more hollow fiber membranes flow in a co-current pattern, a counter-current pattern, a cross-current pattern, or a combination thereof. In some forms, the broth and the solvent flowing axially in one or more hollow fiber membranes flow in a co-current pattern. In some forms, the broth and the solvent flowing axially in one or more hollow fiber membranes flow in a counter-current pattern. In some forms, the broth and the solvent flowing axially in one or more hollow fiber membranes flow in a cross-current pattern.

In some forms, biogas produced in the bioreactor is recirculated into the bioreactor. In some forms, broth is recirculated into the bioreactor.

Generally, a shell side stream flows at a flow rate such that a solvent flowing axially through a plurality of hollow fiber membranes can extract a compound from the shell side stream. In some forms (such as for a 2-L bioreactor) the inlet flow rate is about 2L/day or the hydraulic retention time is about one day.

Further, operational temperature and pH conditions are conducive for compound extraction. In some forms, the inlet stream is provided at a temperature between 4° C. and 35° C., such as 4° C. In some forms, the temperature within the bioreactor is between 28° C. and 35° C. In some forms, pH of the bioreactor is maintained between 5 and 6, such as 5.5.

In some forms, (i) the pH of the bioreactor broth was maintained at 5.5; (ii) the hydraulic retention time was about one day; (iii) and biogas was recirculated every 2 hrs for 5 mins, at a rate of 150 mL/min. Optionally, the temperature of the bioreactor was maintained at about 32° C., such as 32±1° C.

The methods of use provide a process for extracting MCCA, produced by microorganisms in a fermentation reactor by anaerobic fermentation from fermentable biomass, preferably by of liquid-liquid type extraction. The process includes least the steps of bringing an extraction solvent into contact with a fermentation medium and separating the fermentative metabolites from the extraction solvent.

The disclosed bioreactor and methods of use can be further understood through the following enumerated paragraphs or embodiments.

1. A bioreactor containing:

a shell defined by one or more walls and a length, and

a plurality of hollow fiber membranes inside the shell,

wherein the plurality of porous hollow fiber membranes does not span the entire length of the shell.

2. The bioreactor of paragraph 1, wherein between about 10% and about 70%, between about 10% and about 60%, between about 10% and about 50%, between about 20% and about 50%, between about 20% and about 30%, or about 50% of the length of the shell remains unoccupied by the plurality of porous hollow fiber membranes.

3. The bioreactor of paragraph 1 or 2, wherein one end of the plurality of porous hollow fiber membranes is mounted at a first end of the shell and the other end of the plurality of porous hollow fiber membranes is mounted at a second portion of the shell.

4. The bioreactor of any one of paragraphs 1 to 3, wherein one end of the plurality of porous fiber membranes is mounted at a first end of the shell and the other end of the plurality of porous hollow fiber membranes is mounted at about the middle of the shell.

5. The bioreactor of any one of paragraphs 1 to 4, wherein the plurality of porous hollow fiber membranes contains polymeric materials, non-polymeric materials, or a combination thereof.

6. The bioreactor of any one of paragraphs 1 to 5, wherein hollow fiber membranes in the plurality of porous hollow fiber membranes contain cellulose (e.g., regenerated cellulose), cellulose acetate, polysulfone, polyacrylonitrile, inorganic carbon, alumina, polypropylene, polyethylene, polyvinylidene fluoride, polytetrafluoroethylene, polyether sulfone, sulfonated polyether sulfone, or a combination thereof

7. The bioreactor of any one of paragraphs 1 to 6, wherein porous hollow fiber membranes in the plurality of porous hollow fiber membranes are potted at both ends with a material selected from polyepoxides (such as solvent-resistant polyepoxides), polyurethane, polypropylene, or a combination thereof.

8. The bioreactor of any one of paragraphs 1 to 7, wherein the plurality of porous hollow fiber membranes is configured as cylindrical tube bundles, helically wound bundles, rectangular bed of fibers, or a combination thereof.

9. The bioreactor of any one of paragraphs 1 to 8, wherein the shell has a shape selected from a cylinder, rectangle, square, pentagon, hexagon, or octagon.

10. The bioreactor of any one of paragraphs 1 to 9, wherein the shell contains a material selected from polypropylene, polyvinylidene fluoride, polyvinyl chloride, metals (such as silver, zinc, copper, aluminum, nickel, iron, titanium, and chromium), metal alloys of any of the preceding metals, ceramics, glass, borosilicate-tempered glass, steel (e.g., stainless steel, carbon steel, etc), plastics (e.g., epoxy resins, UV cured resins, thermosetting resins, etc), ceramics, composites, quartz, silicon, or a combination thereof

11. The bioreactor of any one of paragraphs 1 to 10, wherein the bioreactor contains biocarriers in the shell volume.

12. The bioreactor of paragraph 11, wherein the biocarriers are selected from granular activated carbon, glass, polystyrene beads, plastic materials of polypropylene, polyethylene, polyvinyl dichloride, polytetrafluoroethylene, latex, rubber, agarose, or a combination thereof.

13. The bioreactor of any one of paragraphs 1 to 12, containing microorganisms.

14. The bioreactor of paragraph 13, wherein the microorganisms are sequestered on the biocarriers, within pore spaces of the biocarriers, or a combination thereof.

15. The bioreactor of paragraph 13 or 14, wherein the microorganisms include active chain-elongation organisms.

16. A method of extracting one or more compounds from a broth, the method involving:

contacting a shell side stream containing the broth with the plurality of porous hollow fiber membranes of the bioreactor of any one of claims 1 to 15.

17. The method of paragraph 16, wherein a solvent flows axially through the plurality of porous hollow fiber membranes.

18. The method of paragraph 17, wherein the shell side stream and solvent flowing axially through the plurality of porous hollow fiber membranes flow in a co-current pattern, a counter-current pattern, or a cross-current pattern, or a combination thereof.

19. The method of paragraph 17 or 18, wherein the shell side stream and the solvent flowing axially through the plurality of porous hollow fiber membranes flow in a co-current pattern.

20. The method of paragraph 18 or 19, wherein the solvent flowing axially through the plurality of porous hollow fiber membranes contains mineral oil solvent with tri-n-octylphosphine oxide (e.g., mineral oil solvent with 3% tri-n-octylphosphine oxide), N-methylpyrrolidone, methyl isobutyl ketone, xylene, n-butanol, 1,2-butanediol, or a combination thereof.

21. The method of any one of paragraphs 18 to 20, wherein the solvent flowing axially through the plurality of porous hollow fiber membranes contains mineral oil solvent with tri-n-octylphosphine oxide (e.g., mineral oil solvent with 3% tri-n-octylphosphine oxide).

22. The method of any one of paragraphs 17 to 21, the method involving:

contacting the solvent that flows axially through the plurality of porous hollow fiber membranes with a pertraction solution after the solvent exits the plurality of porous hollow fiber membranes.

23. The method of paragraph 22, wherein the pertraction solution has an alkaline pH, such as between 8 and 14, between 9 and 13, or between 9 and 11.

24. The method of paragraph 22 or 23, wherein the pertraction solution has a pH between 9 and 11.

25. The method of any one of paragraphs 16 to 24, wherein the bioreactor is maintained at a temperature between 28° C. and 35° C.

26. The method of any one of paragraphs 16 to 25, wherein the shell side stream containing the broth is maintained at a pH between 5 and 6, such as 5.5

27. The method of any one of paragraphs 16 to 26, the method involving:

recirculating biogas through the bioreactor.

28. The method of any one of paragraphs 16 to 27, wherein:

(i) the pH of the bioreactor broth is maintained at 5.5,

(ii) the bioreactor has a hydraulic retention time of about one day, and

(iii) biogas is recirculated every 2 hrs for 5 mins, at a rate of 150 mL/min.

29. The method of any one of paragraphs 16 to 28, wherein the one or more compounds are medium chain carboxylic acids.

### EXAMPLES

### 2. Materials and Methods

### 2.1. Substrate and inoculum

Synthetic basal medium for the biotic experiments was prepared according to a previous study (Kucek, et al., Energy Environ. Sci. 2016b, 9, 3482-3494) with the following exceptions: yeast extract (1 g L-1) and sodium bicarbonate (1 g L-1). Two different concentration ratios of acetate to ethanol were applied during the nine periods to maintain sufficient ethanol in the influent (Table 1).

TABLE 1
Experimental approach and operating conditions for submerged
pertraction bioreactor during Periods I to IX.

| | Date | | | Influent |
|---|---|---|---|---|
| Periods | (Days) | Anti-fouling strategy | Pertraction type | (Ethanol:Acetate)^{a} |
| I | 0-50 | No | Internal | 50:25 |
| II | 51-83 | Biogas recirculation every 4 hrs | Internal | 50:25 |
| | | for 1 min at 40 mL min⁻¹ | | |
| III | 84-132 | Biogas recirculation every 6 hrs | Internal | 50:25 |
| | | for 30 min at 20 mL min⁻¹ | | |
| IV | 133-149 | Biogas recirculation every 6 hrs | Internal | 50:25 |
| | | for 30 min at 80 mL min⁻¹ | | |
| V | 150-223 | Biogas recirculation every 2 hrs | Internal | 50:25 |
| | | for 5 min at 150 mL min⁻¹ | | |
| VI | 224-248 | Biogas recirculation every 2 hrs | Internal | 100:25 |
| | | for 5 min at 150 mL min⁻¹ | | |
| VII | 249-282 | Biogas recirculation every 2 hrs | Internal + external | 100:25 |
| | | for 5 min at 150 mL min⁻¹ | | |
| VIII | 283-374 | Broth recirculation at flow rate | Internal + external | 100:25 |
| | | of 300 mL min⁻¹ | | |
| IX | 375-402 | Broth recirculation at flow rate | Internal + external | 100:25 |
| | | of 1600 mL min⁻¹ | | |

^{a}The ratios are mol:mol.

The pH of the medium was adjusted to 5.50 with 4 M of sodium hydroxide. The synthetic broth was prepared with 3 g L-1 of Na2SO4, 20 mM of acetate, 20 mM of n-butyrate, 10 mM of n-caproate, and 1 mM of n-caproate for the abiotic pertraction experiments. The pH of the synthetic broth was set at 5.5.

The reactor was inoculated with a mixed biomass consisting of mangrove sediments, wastewater sludge, granular sludge and anaerobic digestion sludge to achieve high microbial diversity in the mixed inoculum. The mangrove sediment was collected from the King Abdullah Monument area (Thuwal, Saudi Arabia). The wastewater sludge was collected from the wastewater treatment plant at King Abdullah University of Science and Technology. The granular sludge and anaerobic digestion sludge were derived from a full-scale aerobic granular sludge reactor (Ali, et al., Water Res. 2020, 170, 115345) and lab-scale anaerobic digestion reactor (Cheng, et al., Environ. Int. 2019, 133, 105165). Each of the inoculum sources was washed three times in a basal medium, and 100 mL of each inoculum was added to the bioreactor.

### 2.2. Bioreactor Construction and Pertraction

The up-flow bioreactor contained a cylinder with an internal diameter of 5.5 cm and height of 95 cm (FIG. 1A), and had a working volume of 2.25 L. The temperature of the bioreactor was maintained at 32±1° C. using a recirculating water bath (MP-5H, Hinotek, China). The bioreactor broth pH was maintained at 5.5±0.1 by an automatic pH controller (400 pH/ORP, Cole-Parmar, USA) and a dosing pump to add sodium hydroxide solution (2 M). The biogas was collected and recorded by a flow gas meter (TG05, Ritter, Germany). The synthetic medium was continuously fed to the bioreactor from a refrigerated container (4° C.) using a peristaltic pump, maintaining an HRT of ˜1 day (FIG. 1C). The effluent continuously exited the bioreactor using an overflow pipe fixed near the top of the bioreactor.

MCCAs were continuously extracted from the bioreactor with two types of in-line pertraction: internal and external hollow fiber membrane. For the internal hollow fiber membrane pertraction, 4 hollow fiber membranes (Cleanfil-SMembrane, Kolon Industries, South Korea) 44 cm long each were assembled as a single bundle using polyepoxides (Flow-mix, Devcon, USA). One end of the bundle was connected to the bottom port of the bioreactor. The other end of the hollow fiber bundle was connected to the middle port of the bioreactor. Mineral oil solvent (VWR, USA) with 3% tri-n-octylphosphine oxide (TOPO) (Alfa Aesar, USA) was used as the hydrophobic solvent and it was recycled at an up-flow rate of 1 mL min-1 (Cerampump, Fluid metering, USA) through the hollow fiber membranes from a two-phase reservoir in which 200 mL of the hydrophobic solvent and 250-300 mL of the alkaline pertraction solution were phase-separated (FIG. 1A). The alkaline extraction solution was initially buffered with 0.2 M boric acid and was maintained at a pH of 9-11 with manual addition of 2 M sodium hydroxide solution.

For external hollow fiber membrane pertraction, a forward and a backward membrane models with a contact area of 0.75 m2 (MD063CP2N, Microdyn, Germany) were applied which is similar to those used in a previous study (FIG. 1B) (Xu, et al., Joule 2018, 2, 280-295). The bioreactor broth was continuously circulated through the exterior space of the forward membrane model at a flow rate of 50 mL min-1. A 5 μm pore size filter (GS-6sed/5, Pentek, USA) was placed before the forward membrane model to prevent membrane fouling and was replaced every month. A constant hydrophobic solvent was circulated at a flow rate of 30 mL min-1 through the interior of the forward and backward hollow fiber membrane models. An alkaline pertraction solution (2.5 L) from a well-mixed reservoir was circulated at a flow rate of 40 mL min-1 through the exterior of the backward hollow fiber membrane model. This alkaline pertraction solution was similar to the one used for the internal hollow fiber membrane pertraction.

### 2.3. Experimental Periods

To reduce membrane fouling, two operating strategies were adopted (Table 1): biogas recirculation (Periods II to VII) and broth recycle flow rate (Periods VIII to IX). During Period I (start-up phase), the bioreactor was oerated for 50 days without any anti-fouling treatment. During Periods II to VII, successive cycles of biogas recirculation were varied, including the settling time, flow rate, and time of recirculation (Table 1). During Periods VIII and IX, the bioreactor broth was recirculated to reduce membrane fouling at an upflow rate of 300 mL min-1 (7.6 m h-1) or 1600 mL min-1 (40.5 m h-1) using a gear pump (MG200-400, Fluid-o-Tech, Italy) and a variable frequency drive (JNEV-201-H1FN4S, Teco-Westinghouse, USA). To compare the extraction efficiency between the internal and external hollow fiber membrane, the two types of pertraction were conducted in parallel during Period VII to IX (FIG. 1B, Table 1). Each period was operated for at least 20×HRT, and the average HRT and organic loading rate (OLR) were reported (FIG. 1C).

During the abiotic internal hollow fiber membrane experiments, a carboxylate synthetic solution was continuously fed to the abiotic internal hollow fiber membrane reactor. The mass transfer coefficient, and the effects of the solvent-alkaline solution interfacial area on mass transfer rate were investigated. Two interfacial areas of 62.4 cm2 and 181.8 cm2 were conducted during Stage A and Stage B, respectively. An H-type glass container and a cell culture flask were used for the abiotic pertraction experiment with interfacial areas of 62.4 cm2 and 181.8 cm2, respectively. For the biotic pertraction experiment, only the H-type glass container was used because increasing the interfacial area did not affect the mass transfer rate.

### 2.4. Microbial Community Analysis

Biomass samples for Illumina 16S rRNA gene sequencing analysis were collected from the bioreactor mixed broth during Periods I to IX (Days 25, 68, 110, 137, 211, 247, 277, 325, and 380) with one sample per period. 20 Biomass samples were collected from a sampling port that was located one-third from top of the bioreactor. The bioreactor mixed broth was collected in mL centrifuge tubes and centrifuged at 10,000×g for 10 min to obtain a pellet. The obtained biomass pellets were stored at −80° C. until further analysis.

Genomic DNA extraction, DNA amplification and sequencing were performed according to the protocol in a previous study (Alqahtani, et al., Adv. Funct. Mater. 2021, 28, 1804860). Operational taxonomic unit (OTU) abundance was estimated at 97 identities using the usearch (v. 7.0.1090-usearch_global) (Bian, et al., J. Mater. Chem. A. 2021, 6, 17201-17211). Taxonomy was assigned to representative OTUs using the RDP classifier in QIIME (Caporaso et al. 2010). The following analyses were performed in R (v. 4.0.2) using the ampvis package (v.2.6.4), receiving 377 unique OTUs. Alpha diversity was analyzed using the Shannon diversity index, Simpson index and invSimpson index. Heatmap was created to represent the top 20 OTU using the ggplot package in R.

### 2.5. Liquid Sampling, Analytical Procedures, and Calculations

The bioreactor broth samples were collected every other day directly from the sampling port. The samples were filtered through a 0.22-μm pore filter prior to the analyses of carboxylic acids and ethanol. The composition of carboxylic acids and ethanol was determined with a gas chromatograph GC) (6890A Series, Agilent Technologies Inc., USA) as described previously (Usack and Angenent, Water Res. 2015, 87, 446-457). The concentrations of methane, carbon dioxide, and hydrogen in the biogas were measured weekly using a GC (model 310C; SRI Instruments, USA) as previously described (Alqahtani, et al., Adv. Funct. Mater. 2021, 28, 1804860). Detailed information on calculations is provided in the below (Eq. S1-S4).

### EQUATIONS

### Product Transfer Rate (mmol m⁻² d⁻¹):

$$\frac{m}{S} \qquad ( Eq . S1 )$$

where:

m=slope of the increasing specific carboxylate in the pertraction solution against time, mmol d⁻¹

S=area of hollow fiber membrane, m²

### Volumetric Production Rate (mmol C L⁻¹ d⁻¹):

$$( \frac{{C}_{e , n} V}{H R T} = {m}_{i} + {m}_{e} ) \frac{M}{V} \qquad ( Eq . S2 )$$

where:

C_{e,n}=concentration of carboxylic acid in the effluent on day n, mM

V=volume of the reactor, L

HRT=hydraulic retention time on day n, d

mᵢ=slope of the increasing specific carboxylate in the pertraction solution using internal hollow fiber against time, mmol d⁻¹

mₑ=slope of the increasing specific carboxylate in the pertraction solution using external hollow fiber against time, mmol d⁻¹

M=conversion factor from mmol to mmol C; for example, acetic acid was 2

### Conversion Efficiency into Methane (%, mM C/mM C)

$$\frac{{P}_{m}}{{L}_{a} + {L}_{e}} \qquad ( Eq . S3 )$$

where:

Pₘ=methane production rate, mM C d⁻¹

Lₐ=acetate loading rate, mM C d⁻¹

Lₑ=ethanol loading rate, mM C d⁻¹

### Carboxylates Extraction Rates by Hollow Fiber Membrane (mmol m⁻² d⁻¹)

$$\frac{{C}_{e}}{M} \qquad ( Eq . S4 )$$

where:

Cₑ=specific carboxylic acid extraction rate in the extraction solution, mmol d⁻¹

M=area of hollow fiber membrane, m⁻²

### 3. Results and Discussion

### 3.1. Operation of Internal Hollow Fiber Model with Abiotic Synthetic Broth

The objective of this work was to demonstrate the technical feasibility of utilizing a submerged (i.e., internal) hollow fiber membrane model in the bioreactor for MCCA extraction. The use of external hollow fiber membrane model for pertraction has been previously applied where a hydrostatic pressure of 0.5-3 psi has been used successfully in the broth side of the membrane by adjusting the valve to prevent organic solvent transferring into the fermentation broth (Kucek, et al., Water Res. 2016a, 93, 163-171; Kucek, et al., Energy Environ. Sci. 2016b, 9, 3482-3494; Kucek, et al., Front. Microbiol. 2016c, 7, 1892; Xu, et al., Chem. Commun. 2015, 51, 6847-6850; Xu, et al., Joule 2018, 2, 280-295). However, it is difficult to apply a hydrostatic pressure in the atmospheric bioreactor in the case of a submerged hollow fiber membrane model in the bioreactor. To circumvent this problem, the hollow fiber membrane model was placed at the middle-to-bottom of the bioreactor (hydrostatic pressure: 0.7 psi to 1.3 psi, FIGS. 1A-1B). Steady operation was successfully achieved using abiotic synthetic broth.

Several factors affect the steady-state operation of pertraction system and the extraction rate of MCCAs, including forward and backward contactor area, the flow rate of organic solvent and alkaline solution, type of organic solvent, etc (Kucek, et al., Energy Environ. Sci. 2016b, 9, 3482-3494; Saboe, et al., Green. Chem. 2018, 20, 1791-1804). MCCA extraction by pertraction included two steps: 1) MCCAs transferring from broth to organic solvent (forward); and 2) MCCAs transferring from organic solvent to extraction solution (backward). In the backward MCCA extraction in a 15 pertraction system, an alkaline extraction solution is used to supply a gradient as a driving force for extraction (Xu, et al., Environ. Sci. Technol. 2021, 55, 634-644). In the current study, two phases of alkaline extraction solution and organic solvent contacted directly without any membrane separator for backward extraction (FIG. 1A). To determine whether the step of backward MCCA extraction limits the mass transfer in the pertraction system, two contactor area of 62.4 cm2 and 181.8 cm2 were applied in Stage A and B, respectively. In Stage A, the stable mass transfer of acetate, n-butyrate, n-caproate, and n-caprylate were obtained at an extraction rate of 2.3, 5.2, 13.7 and 6.3 mmol m-2 d-1, respectively (FIG. 1D). Increasing the 25 contactor area to 181.8 cm2 in Stage B did not affect the carboxylate extraction rates (FIG. 1D), indicating that the contactor area of 62.4 cm2 for alkaline extraction solution and organic solvent was large enough for this pertraction system. Indeed, in a previous study it has been reported that the process of backward extraction was not the limiting step when using the same contactor area of forward and backward extraction. (Kucek, et al., Energy Environ. Sci. 2016b, 9, 3482-3494).

Utilizing a more apolar solvent can selectively extract longer carbon chain carboxylic acids and avoids the removal of SCCAs, which are used as a carbon source for chain elongation. In this study, a mixture of mineral oil (apolar) and 3% TOPO (polar) was used as organic solvent, which has been previously applied to extract MCCAs from fermentation reactor (Carvajal-Arroyo, et al., Chem. Eng. J. 2020, 416, 127886; Ge, et al., Environ. Sci. Technol. 2015, 49, 8012-8021; Kucek, et al., Energy Environ. Sci. 2016b, 9, 3482-3494; Urban, et al., Energ. Environ. Sci. 2017, 10, 2231-2244; Xu, et al., Chem. Commun. 2015, 51, 6847-6850; Xu, et al., Environ. Sci. Technol. 2021, 55, 634-644; Xu, et al., Joule 2018, 2, 280-295). The mineral oil has low toxicity and a food-grade of it can be used in the food industry (Saboe, et al., Green. Chem. 2018, 20, 1791-1804). It was observed that mineral oil mixed in the fermentation bioreactor for a short period did not affect the conversion processes of substrate to MCCAs. The high viscous mineral oil can lower the risk of organic solvent transferring into the bioreactor. Although higher partition coefficients of the solvents for MCCAs such as propiophenone and 2-undecanone, were previously observed (Saboe, et al., Green. Chem. 2018, 20, 1791-1804), it probably has a negative impact on conversion of substrate to MCCAs in the fermentation bioreactor once these solvents dissolved in the bioreactor. The addition of TOPO as an extractant can achieve a high equilibrium constant and increases the solvent affinity for carboxylic acid due to the polarity of its P-O bond (Carvajal-Arroyo, et al., Chem. Eng. J. 2020, 416, 127886; Saboe, et al., Green. Chem. 2018, 20, 1791-1804).

### 3.2. The Effect of Anti-Membrane Fouling Strategies on MCCA Production Rate and Extraction Rate by Internal Hollow Fiber Membrane

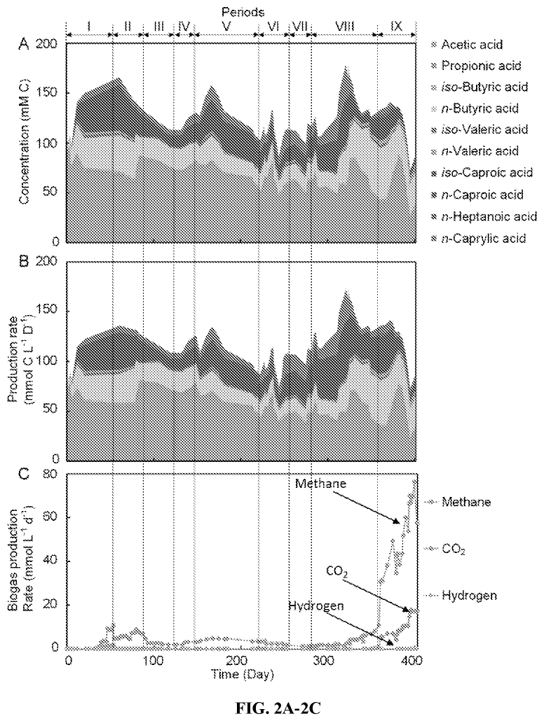

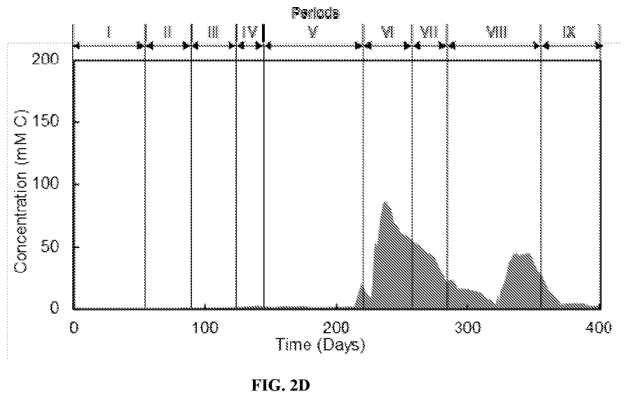

Two antifouling strategies were evaluated in this work, i.e., periodic biogas recirculation (Period II to VII) and broth recirculation (Period VIII to IX) (Table 1). During Period I, with no introduction of antifouling strategy, an extraction rate of 16.7±8.7 for n-caproate and 9.7±0.9 mmol m-2 d-1 for n-caprylate by internal pertraction was achieved. Introducing periodic biogas recirculation in Period II, resulted in an unexpected decrease in MCCA extraction rates to 11.1±1.4 and 6.3±3.5 mmol m-2 d-1 for n-caproate and n-caprylate, respectively. Several factors might have been responsible for this decrease in MCCA extraction rate as explained below. The volatile solids (VS) decreased from 5.2±0.2 to 3.9±0.01 g L−1 (FIG. 1E) due to biomass washout when biogas recirculation was applied, and this in turn might have resulted in the decrease of the concentration of n-caproate (from 31.4±13.0 to 18.2±8.9 mM C) and n- caprylate (from 6.2±2.1 to 5.2±0.4 mM C) (FIG. 2A). Decrease in MCCA extraction rate has been previously observed when the concentration of undissociated MCCAs decreased in the fermentation broth (Carvajal-Arroyo, et al., Chem. Eng. J. 2020, 416, 127886; Kucek, et al., Water Res. 2016a, 93, 161-171). The VS decrease also resulted in the decrease in MCCA production from 35.9±13.4 mmol C L−1 d−1 during Period Ito 23.4 ±9.1 mmol C L−1 d−1 during Period (Table 2.).

TABLE 2
Bioreactor production rate and conversion efficiency during the
Periods I to IX with internal and external hollow fiber membrane.

| | Period | Period | Period | Period | Period | Period | Period | Period | Period |
|---|---|---|---|---|---|---|---|---|---|
| | I | II | III | IV | V | VI | VII | VIII | IX |
| Volumetric | 81.3 ± | 92.6 ± | 95.2 ± | 95.2 ± | 84.7 ± | 165.3 ± | 157.5 ± | 166.7 ± | 178.6 ± |
| EthOH¹ | 7.3 | 1.7 | 1.8 | 0.9 | 4.3 | 8.2 | 6.2 | 15.3 | 12.7 |
| loading rate | | | | | | | | | |
| (mmol C L⁻¹ | | | | | | | | | |
| d⁻¹) | | | | | | | | | |
| Volumetric | 40.7 ± | 46.3 ± | 47.6 ± | 42.4 ± | 41.3 ± | 39.4 ± | 41.7 ± | 41.7 ± | 44.6 ± |
| Ac¹ loading | 3.6 | 0.9 | 0.5 | 2.2 | 2.0 | 1.6 | 3.8 | 3.8 | 3.2 |
| rate | | | | | | | | | |
| (mmol C L⁻¹ | | | | | | | | | |
| d⁻¹) | | | | | | | | | |
| EthOH in | 0.7 ± | 0.7 ± | 1.7 ± | 2.7 ± | 13.9 ± | 62.1 ± | 34.1 ± | 23.3 ± | 3.5 ± |
| effluent | 0.2 | 0.4 | 0.5 | 0.3 | 3.3 | 7.4 | 10.1 | 13.4 | 0.7 |
| (mmol C L⁻¹ | | | | | | | | | |
| d⁻¹) | | | | | | | | | |
| CH₄ | 6.9 ± | 7.8 ± | 2.1 ± | 2.7 ± | 3.9 ± | 2.4 ± | 1.5 ± | 36.1 ± | 68.1 ± |
| production | 3.1 | 0.8 | 0.2 | 0.7 | 0.6 | 0.05 | 0.5 | 8.0 | 6.8 |
| rate (mmol C | | | | | | | | | |
| L⁻¹ d⁻¹) | | | | | | | | | |
| EthOH + Ac- | 5.6 | 5.6 | 1.4 | 1.8 | 3.1 | 1.1 | 0.7 | 17.3 | 30.5 |
| into-CH4 | | | | | | | | | |
| efficiency | | | | | | | | | |
| (% mmol C) | | | | | | | | | |
| CO₂ | 1.0 ± 0.4 | 0.05 ± | 0.01 ± | 0.01 ± | 0.05 ± | 0.03 ± | 0.01 ± | 5.9 ± | 17.2 ± |
| production | | 0.006 | 0.004 | 0.001 | 0.009 | 0.006 | 0.001 | 1.0 | 0.3 |
| rate (mmol C | | | | | | | | | |
| L⁻¹ d⁻¹) | | | | | | | | | |
| CA¹ | 127.5 ± | 113.3 ± | 116.3 ± | 118.8 ± | 89.4 ± | 84.5 ± | 107.2 ± | 138.8 ± | 90.6 ± 21.6 |
| production | 24.6 | 26.7 | 14.9 | 11.3 | 10.7 | 12.8 | 13.4 | 22.1 | |
| rate (mmol C | | | | | | | | | |
| L⁻¹ d⁻¹) | | | | | | | | | |
| MCCA¹ | 35.9 ± | 23.4 ± | 21.0 ± | 25.7 ± | 28.0 ± | 27.5 ± | 46.5 ± | 52.7 ± | 20.4 ± |
| Volumetric | 13.4 | 9.1 | 7.0 | 4.8 | ±7.1 | 6.2 | 6.8 | 6.3 | 9.3 |
| production | | | | | | | | | |
| rate (mmol C | | | | | | | | | |
| L⁻¹ d⁻¹) | | | | | | | | | |

¹EthOH: ethanol; AC: acetate; CA: carboxylic acid; MCCA: medium chain carboxylic acid

Periodic biogas sparging (Table 1) was continued during Period III to Period V and the highest MCCA extraction rate of 39.5 mmol m−2 d−1 was obtained during Period IV. During Period IV, the operation of biogas recirculation every 6 hr for 30 min at a flow rate of 80 min min-1 and ethanol:acetate of 50:25 (mol:mol) was considered the optimum condition for MCCA extraction in this system. Biogas recirculation was applied in submerged membrane system not only to scour the outer membrane surface and induce a shear force at the membrane surface to remove the accumulated foulants (Fulton, et al., Desalination 2011, 281, 128-141; Vermaas, et al., Environ. Sci. Technol. 2014, 48, 3065-3073), but also to induce a turbulent flow which can increase mass transfer rate (Laptev, et al., J. Eng. Phys. Thermophy. 2015, 88, 207-213.). Mathematical modelling analysis should be used in future studies to describe and understand the effect of the performing conditions on the process of mass transfer.

To increase the MCCA production rate, the influent concentration of ethanol was doubled to 100 mM during Period VI (Table 1). Enough ethanol (75.2±8.9 mM C) was present in the broth as an electron donor and carbon source to sustain a promising chain elongation rate (FIG. 2D). The average MCCA extraction rate of 97.4 mmol m-2 d−1 (62.0±6.0 mmol C6 m−2 d−1 and 35.4±6.3 mmol C8 m−2 d−1) obtained here were higher than the rates reported in previous chain elongation studies using external membrane pertraction (Table 3).

TABLE 3
Performance parameters and MCCA extraction rates of external
membrane model pertraction system from previously published studies
which were similar to the one used in the present study.

| | | Ratio of | | n- | n- | |
|---|---|---|---|---|---|---|
| | | membrane | Broth | Caproate | Caprylate | MCCAs |
| | | area to | recycle | extraction | extraction | extraction |
| | Type | reactor | flow | rate | rate | rate |
| Refer- | of the | volume | rate (m | (mmol | (mmol m⁻² | (mmol |
| ence | broth | (m² L⁻¹) | hr⁻¹) | m⁻² d⁻¹) | d⁻¹) | m⁻² d⁻¹) |
| 6 | Filtered | 2.5 | 1.1 | 5.3 | 0.7 | 6.8 |
| | bioreactor | | | | | |
| | broth | | | | | |
| 5 | Filtered | 0.4 | 1.0 | 11.4 | 11.1 | 22.5 |
| | bioreactor | | | | | |
| | broth | | | | | |
| 4 | Abiotic | 11.6 | 9.5 | 6.6 | — | — |
| | synthetic | | | | | |
| | broth | | | | | |
| 4 | Filtered | 2.0 | 3.4 | 3.0 | 27.2 | 30.2 |
| | bioreactor | | | | | |
| | broth | | | | | |
| 1 | Filtered | 0.14 | 1.9 | 57.8 | — | 11.1 |
| | bioreactor | | | | | g m⁻² |
| | broth | | | | | d⁻¹ |
| 3 | Filtered | 2.5 | 1.6 | 10.5 | — | 10.5 |
| | bioreactor | | | | | |
| | broth | | | | | |
| 2 | Filtered | 1.6 | — | 17.7 | — | 17.7 |
| | bioreactor | | | | | |
| | broth | | | | | |

Carvajal-Arroyo et al (Carvajal-Arroyo, et al., Chem. Eng. J. 2020, 416, 127886) reported a MCCA mass flux of 11.1 g m−2 d−1 (the aggregated reported number of MCCAs does not allow comparison with a molar unit), while an average maximum MCCA mass flux of 12.2 g m−2 d−1 was achieved in the present study which was slightly higher than that obtained in Carvajal-Arroyo et al. A regular offline cleaning (once every 3-5 weeks) for the external membrane model by flushing with water to remove the accumulated foulants was performed in previous studies (Xu, et al., Chem. Commun. 2015, 51, 6847-6850; Xu, et al., Environ. Sci. Technol. 2021, 55, 634-644), while a continuous high extraction rate was achieved in this work at least for 248 days by regularly recirculating without any offline washing or application of anti-fouling chemical agents. The broth biomass concentration in this work (FIG. 1E) was relatively lower than most previous studies (Ge, et al., Environ. Sci. Technol. 2015, 49, 8012-8021; Xu, et al., Joule 2018, 2, 280-295), and it has been reported that increase in biomass could result in an increase in conversion rate and concentration of products (Xu, et al., Joule 2018, 2, 280-295), thus further leading to high extraction rate (Carvajal-Arroyo, et al., Chem. Eng. J. 2020, 416, 127886; Kucek, et al., Water Res. 2016a, 93, 163-171). Therefore, increasing the biomass concentration (e.g., adding carriers) would result in further increase in MCCA extraction rate using internal pertraction, albeit probably negatively affecting membrane fouling.

During Period VIII, the broth was recirculated at a flow rate of 300 ml min-1 (upflow velocity of 7.6 m h-1) to reduce hollow fiber membrane fouling with an internal and external hollow fiber membrane pertraction operated in parallel (Table 1). A higher MCCA extraction rate (31.2 mmol m−2 d−1) was observed in Period VIII (broth recirculation with two types of pertraction operated in parallel) compared to 24.3 mmol m−2 d−1 during Period VII (biogas recirculation with two types of pertraction operated in parallel) (Table 4).

TABLE 4
Carboxylates extraction rates with two pertraction
strategies during the nine periods.

| | | | | | |
|---|---|---|---|---|---|
| | Acetate | n-Butyrate | n-Caproate | n-Caprylate | MCCAs |
| | extraction | extraction | extraction | extraction | extraction |
| | rate (mmol | rate (mmol | rate (mmol | rate (mmol | rate (mmol |
| Periods | m⁻² d⁻¹) | m⁻² d⁻¹) | m⁻² d⁻¹) | m⁻² d⁻¹) | m⁻² d⁻¹) |

Internal pertraction

| | | | | | |
|---|---|---|---|---|---|
| I | 9.7 ± 1.2 | 11.2 ± 5.2 | 16.7 ± 8.7 | 9.7 ± 0.9 | 26.4 ± 9.6 |
| II | 2.6 ± 1.1 | 1.3 ± 0.4 | 11.1 ± 1.4 | 6.3 ± 3.5 | 17.4 ± 4.9 |
| III | 7.3 ± 3.1 | 6.0 ± 1.4 | 20.3 ± 7.7 | 6.7 ± 0.3 | 27.0 ± 8.0 |
| IV | 6.1 ± 0.13 | 8.8 ± 1.1 | 20.6 ± 7.4 | 18.9 ± 4.2 | 39.5 ± 11.6 |
| V | 1.6 ± 0.5 | 1.6 ± 0.6 | 14.8 ± 2.2 | 6.1 ± 1.4 | 20.9 ± 3.6 |
| VI | 9.6 ± 5.9 | 14.3 ± 7.6 | 62.0 ± 6.0 | 35.4 ± 6.3 | 97.4 ± 12.3 |
| VII | 5.9 ± 2.0 | 5.9 ± 1.8 | 14.0 ± 8.0 | 10.3 ± 5.5 | 24.3 ± 13.5 |
| VIII | 7.3 ± 1.4 | 7.3 ± 0.8 | 18.7 ± 6.2 | 12.5 ± 3.7 | 31.2 ± 9.9 |
| IX | 3.0 ± 1.5 | 3.8 ± 3.0 | 5.0 ± 1.9 | 10.0 ± 1.7 | 15.0 ± 3.6 |

External pertraction

| | | | | | |
|---|---|---|---|---|---|
| VII | — | 0.56 ± 0.14 | 5.7 ± 0.9 | 2.0 ± 0.2 | 7.7 ± 1.1 |
| VIII | 0.2 ± 0.02 | 1.1 ± 0.1 | 9.4 ± 1.1 | 1.0 ± 0.1 | 10.4 ± 1.2 |
| IX | 0.14 ± 0.03 | 0.33 ± 0.08 | 2.0 ± 0.6 | 1.2 ± 0.4 | 3.2 ± 1.0 |

The results indicated that the extraction rate during operation with broth recirculation was higher than operation with biogas recirculation when internal and external membrane pertraction were operated in parallel. Even though similar fouling strategy (biogas recirculation rate and frequency) was applied in Period VI and VII and similar ethanol:acetate ratio (mol:mol), the extraction rate was significantly higher in Period VI (97.4. mmol m−2 d−1) than Period VII (24.3 mmol m−2 d−1), possibly because only internal hollow fiber membrane pertraction (1.5% membrane area of external membrane area) was applied in Period VI compared to internal and external in Period VII. Increasing the broth recirculation rate to 1600 mL min-1 (40.5 m h-1) in Period IX resulted in an obvious decrease in MCCA extraction rate to 15.0 mmol m-2 d-1. Under a higher broth up-flow velocity, the concentrations of n-caproate and n-caprylate in the broth decreased to 7.6 mM C and 3.9 mM C, respectively (FIG. 2A). The decrease in extraction rate could be due to the low MCCA concentration in the broth, where more substrates were converted to methane than MCCA (FIG. 2C).

### 3.3. Comparison of Internal and External Pertraction on MCCA Extraction Rate and Production Rate

To evaluate the extraction efficiency of internal pertraction, an external pertraction was set up and operated in parallel with internal pertraction to extract MCCAs from the fermentation reactor during Period VII to IX. During these periods, the extraction rate of n-caproate and n-caprylate by internal pertraction was 2.0- to 2.5-fold and 5.2- to 12.5-fold higher than by external pertraction, respectively (Table 4). The results indicated that the MCCA extraction efficiency by internal pertraction was much higher than by external pertraction with the same chain elongation bioreactor (FIG. 1B). It has been reported that MCCA mass transfer limitations were at the interface of the fermentation broth and the hydrophobic membrane contactor in the external pertraction system, which was similar to the one used in the present work, and increasing the recycle flow rate of broth increased MCCA mass transfer (Kucek, et al., Energy Environ. Sci. 2016b, 9, 3482-3494). In the current study, a broth recycle flow rate of 3 L hr-1 (1.6 m hr-1) was applied in the external pertraction -3.4 m hr-1) used previous studies (Carvajal-Arroyo, et al., Chem. Eng. J. 2020, 416, 127886; Kucek, et al., Energy Environ. Sci. 2016b, 9, 3482-3494; Xu, et al., Environ. Sci. Technol. 2021, 55, 634-644; Xu, et al., Joule 2018, 2, 280-295). In the internal pertraction system, both biogas recirculation 25 (Period VII) and broth recirculation (flow rate of 300 ml min-1 in Period

VIII and 1600 mL min-1 in Period IX) were used to minimize fouling of the membrane and increase mass transfer. Thus, biogas recirculation or higher broth recirculation flow rate could result in higher MCCA mass transfer from the fermentation broth to the hydrophobic solvent. The lower footprint and energy consumption are additional advantages of using internal pertraction system compared to external pertraction system, which requires heating of the recycle broth from external membrane The MCCA production rate was increased from 27.5 mmol C L-1 d-1 during Period VI (biogas recirculation only) to 46.5 mmol C L-1 d-1 during Period VII (broth recirculation only), and the highest production rate of 52.7 mmol C L-1 d-1 was obtained during Period VIII (Table 2).

These results indicate that continuous pertraction can lead to an increase in MCCA production rate due to reducing the MCCA cell toxicity and end-product feedback inhibition in the fermentation bioreactor.

The ratio of pertraction membrane area-to-reactor volume for internal pertraction was only 0.004 m2 L-1, which was much lower than the ratio (0.35 to 2.5 m2 L-1) for external pertraction reported in previous studies

(Kucek, et al., Water Res. 2016b, 93, 163-171; Xu, et al., Environ. Sci. Technol. 2021, 55, 634-644; Xu, et al., Joule 2018, 2, 280-295). In the current study, the MCCA extraction efficiency using internal pertraction was only 0.5-3.8% during all periods. Therefore, the ratio of pertraction membrane area-to-reactor volume was increased to 0.33 m2 L-1 by operating an external pertraction model in parallel with the internal pertraction system during Period VII. The MCCA production rate was increased from 27.5 mmol C L-1 d-1 during Period VI (biogas recirculation only, no external pertraction) to 46.5 mmol C L-1 d-1 during Period VII (biogas recirculation only, with external pertraction), and the highest production rate of 52.7 mmol

C L-1 d-1 was obtained during Period VIII (broth recirculation only) (FIG. 2B; Table 2). These results indicate that continuous pertraction can lead to an increase in MCCA production rate due to reducing the MCCA cell toxicity and end-product feedback inhibition in the fermentation bioreactor.

### 3.4. The Effect of Anti-Membrane Fouling Strategies on

Biomass Concentration and Microbial Community Composition

In the current study, the VS concentration in the fermentation bioreactor decreased from 5.2±0.2 to 3.9±0.01 g L−1 (FIG. 1E) when biogas recirculation was applied during Period II. The VS remained stable at 3.6-4.0 g L−1 during Period II to VII with different biogas recirculation frequency, duration, and flow rate. The VS concentration significantly decreased from 3.6±1.1 g L-1 to 1.5±0.2 g L-1 when broth recirculation rate of 300 ml min-1 (Period VIII) was applied. High biomass concentration in the fermentation bioreactor is commonly considered to achieve high production rates (Carvajal-Arroyo, et al., Green. Chem. 2019, 21, 1330-1339). High concentration of biomass in the chain elongation reactor can be achieved by i) using packing material or settlers (Grootscholten, et al., Bioresour. Technol. 2013, 136, 735-738; Kucek, et al., Energy Environ. Sci. 2016b, 9, 3482-3494; Liu, et al., Water Res. 2017, 119, 150-159); ii) forming a chain elongation granular sludge (Carvajal-Arroyo, et al., Green. Chem. 2019, 21, 1330-1339; Roghair, et al., Process Biochem. 2016, 51, 1594-1598); and iii) using a membrane to prevent biomass washout (Kim, et al., Bioresour. Technol. 2018, 270, 498-503). Therefore, improving reactor design to enhance biomass retention would result in a higher production rates, however, higher biomass concentration might enhance membrane fouling and future studies should evaluate the maximum biomass concentration required to achieve good production rate and MCCA extraction by internal hollow fiber membrane without elevating membrane fouling.

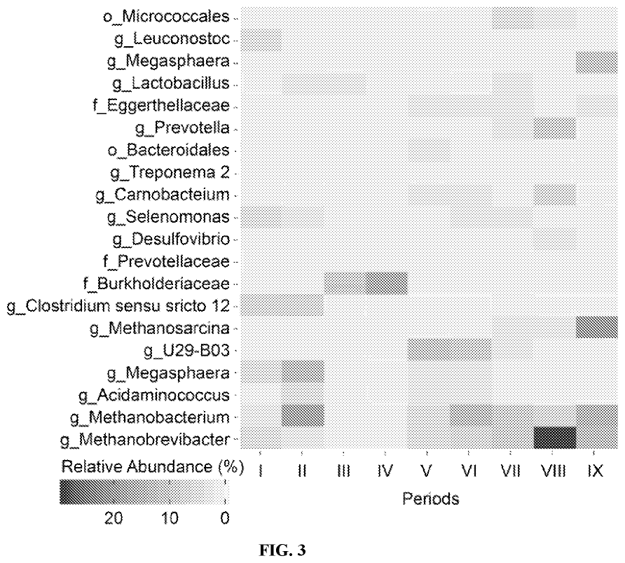

Methanogens exist in nearly every conceivable anaerobic environment and organisms can convert organic substrates effectively into methane because it has the lowest free energy content per electron (Angenent, et al., Environ. Sci. Technol. 2016, 50, 2796-2810; Zinder, “Physiological Ecology of Methanogens,” in Methanogenesis: Ecology, Physiology, Biochemistry & Genetics. Editor J. G. Ferry (Boston, MA: Springer), 1993, 128-206). To establish an MCCA production process in open culture fermentations, one successful option for inhibition of methanogenic activity was maintaining an acidic pH of approximately 5.5 in the fermentation broth (Kucek, et al., Front. Microbiol. 2016c, 7, 1892; Xu, et al., Chem. Commun. 2015, 51, 6847-6850; Xu, et al., Joule 2018, 2, 280-295). In the current study, the pH was maintained at 5.5 and conversion efficiency into methane was low (0.7-5.6% of ethanol and acetate conversion into methane, mol C/mol C; FIG. 2C; Table 2; Eq. S3) during Period Ito Period VII (biogas recirculation) despite the fact that members of hydrogenotrophic methanogens belonging to the genus Methanobacterium and Methanobrevibacter were predominant (accounting for 9.0-19.9% of the total reads) (FIG. 3). During Period VIII (broth recirculation at an up-flow velocity of 7.6 m hr-1), members of the genus Methanobrevibacter (relative abundance of 28.9%), Prevotella (relative abundance of 9.0%) and Methanobacterium (relative abundance of 6.7%) were the predominant OTUs detected in the bioreactor (FIG. 3). The conversion efficiency to methane increased to 17.3% (mol C/mol C, Table 2) in Period VIII. When the broth up-flow velocity was increased to 40.5 m hr-1 (Period IX), methane production rate significantly increased in the biogas (FIG. 2C) and conversion efficiency to methane increased to 30.5% (mol C/mol C, Table 2). It should be noted that the highest relative abundance (46.7%) of methanogens (Methanobrevibacter, Methanobacterium, and Methanosarcina) was detected in Period IX. These results indicate that at high upflow velocity the microbial community shifted towards methanogens. Different bacterial cells experience a different response to physical force (Dufrêne and Persat 2020). Therefore, it was hypothesized that chain elongation microbes were more sensitive to mechanical force generated by fluid flow and pressure as well as surface contact compared to methanogens, and this resulted in the shift in the bioprocess towards methanogenesis.

### Conclusions

A submerged hollow fiber membrane (internal) in the fermentation bioreactor was able to achieve high MCCA extraction rate for a long period by biogas recirculation without any offline washing or anti-fouling chemical agent application to remove foulants. However, higher broth up-flow velocity led to low concentration of MCCAs in the fermentation broth because of shift in conversion towards methane production. The results obtained here showed that the extraction rate of MCCAs by internal pertraction was much higher than by external pertraction (traditional pertraction) in the same bioreactor. The results in this work showed that the concentration of biomass in this system was relatively low. The use of biocarriers may also help in reducing membrane biofouling.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A bioreactor comprising:
a shell defined by one or more walls and a length, and
a plurality of hollow fiber membranes inside the shell,
wherein the plurality of porous hollow fiber membranes does not span the entire length of the shell.

2. The bioreactor of claim 1, wherein between about 10% and about 70%, between about 10% and about 60%, between about 10% and about 50%, between about 20% and about 50%, between about 20% and about 30%, or about 50% of the length of the shell remains unoccupied by the plurality of porous hollow fiber membranes.

3. The bioreactor of claim 1 or 2, wherein one end of the plurality of porous hollow fiber membranes is mounted at a first end of the shell and the other end of the plurality of porous hollow fiber membranes is mounted at a second portion of the shell.

4. The bioreactor of any one of claims 1 to 3, wherein one end of the plurality of porous fiber membranes is mounted at a first end of the shell and the other end of the plurality of porous hollow fiber membranes is mounted at about the middle of the shell.

5. The bioreactor of any one of claims 1 to 4, wherein the plurality of porous hollow fiber membranes comprises polymeric materials, non-polymeric materials, or a combination thereof.

6. The bioreactor of any one of claims 1 to 5, wherein porous hollow fiber membranes in the plurality of porous hollow fiber membranes comprise cellulose (e.g., regenerated cellulose), cellulose acetate, polysulfone, polyacrylonitrile, inorganic carbon, alumina, polypropylene, polyethylene, polyvinylidene fluoride, polytetrafluoroethylene, polyether sulfone, sulfonated polyether sulfone, or a combination thereof.

7. The bioreactor of any one of claims 1 to 6, wherein porous hollow fiber membranes in the plurality of porous hollow fiber membranes are potted at both ends with a material selected from polyepoxides (such as solvent-resistant polyepoxides), polyurethane, polypropylene, or a combination thereof.

8. The bioreactor of any one of claims 1 to 7, wherein the plurality of porous hollow fiber membranes is configured as cylindrical tube bundles, helically wound bundles, rectangular bed of fibers, or a combination thereof.

9. The bioreactor of any one of claims 1 to 8, wherein the shell has a shape selected from a cylinder, rectangle, square, pentagon, hexagon, or octagon.

10. The bioreactor of any one of claims 1 to 9, wherein the shell comprises a material selected from polypropylene, polyvinylidene fluoride, polyvinyl chloride, metals (such as silver, zinc, copper, aluminum, nickel, iron, titanium, and chromium), metal alloys of any of the preceding metals, ceramics, glass, borosilicate-tempered glass, steel (e.g., stainless steel, carbon steel, etc), plastics (e.g., epoxy resins, UV cured resins, thermosetting resins, etc), ceramics, composites, quartz, silicon, or a combination thereof.

11. The bioreactor of any one of claims 1 to 10, wherein the bioreactor comprises biocarriers in the shell volume.

12. The bioreactor of claim 11, wherein the biocarriers are selected from granular activated carbon, glass, polystyrene beads, plastic materials of polypropylene, polyethylene, polyvinyl dichloride, polytetrafluoroethylene, latex, rubber, agarose, or a combination thereof.

13. The bioreactor of any one of claims 1 to 12, comprising microorganisms.

14. The bioreactor of claim 13, wherein the microorganisms are sequestered on the biocarriers, within pore spaces of the biocarriers, or a combination thereof.

15. The bioreactor of claim 13 or 14, wherein the microorganisms comprise active chain-elongation organisms.

16. A method of extracting one or more compounds from a broth, the method comprising:
contacting a shell side stream containing the broth with the plurality of porous hollow fiber membranes of the bioreactor of any one of claims 1 to 15.

17. The method of claim 16, wherein a solvent flows axially through the plurality of porous hollow fiber membranes.

18. The method of claim 17, wherein the shell side stream and solvent flowing axially through the plurality of porous hollow fiber membranes flow in a co-current pattern, a counter-current pattern, or a cross-current pattern, or a combination thereof.

19. The method of claim 17 or 18, wherein the shell side stream and the solvent flowing axially through the plurality of porous hollow fiber membranes flow in a co-current pattern.

20. The method of claim 18 or 19, wherein the solvent flowing axially through the plurality of porous hollow fiber membranes comprises mineral oil solvent with tri-n-octylphosphine oxide (e.g., mineral oil solvent with 3% tri-n-octylphosphine oxide), N-methylpyrrolidone, methyl isobutyl ketone, xylene, n-butanol, 1,2-butanediol, or a combination thereof.

21. The method of any one of claims 18 to 20, wherein the solvent flowing axially through the plurality of porous hollow fiber membranes comprises mineral oil solvent with tri-n-octylphosphine oxide (e.g., mineral oil solvent with 3% tri-n-octylphosphine oxide).

22. The method of any one of claims 17 to 21, the method comprising:
contacting the solvent that flows axially through the plurality of porous hollow fiber membranes with a pertraction solution after the solvent exits the plurality of porous hollow fiber membranes.

23. The method of claim 22, wherein the pertraction solution has an alkaline pH, such as between 8 and 14, between 9 and 13, or between 9 and 11.

24. The method of claim 22 or 23, wherein the pertraction solution has a pH between 9 and 11.

25. The method of any one of claims 16 to 24, wherein the bioreactor is maintained at a temperature between 28° C. and 35° C.

26. The method of any one of claims 16 to 25, wherein the shell side stream containing the broth is maintained at a pH between 5 and 6, such as 5.5

27. The method of any one of claims 16 to 26, the method comprising:
recirculating biogas through the bioreactor.

28. The method of any one of claims 16 to 27, wherein:
(i) the pH of the bioreactor broth is maintained at 5.5,
(ii) the bioreactor has a hydraulic retention time of about one day, and
(iii) biogas is recirculated every 2 hrs for 5 mins, at a rate of 150 mL/min.

29. The method of any one of claims 16 to 28, wherein the one or more compounds are medium chain carboxylic acids.
